(19) United States
(12) Patent Application Publication
(10) Pub. No.: US 2013/0066225 A1
(43) Pub. Date: Mar. 14, 2013

(54) MONITORING INCENTIVE SPIROMETRY

(71) Applicant: Kourosh Kojouri, San Jose, CA (US)

(72) Inventor: Kourosh Kojouri, San Jose, CA (US)

(21) Appl. No.: 13/228,438

(22) Filed: Sep. 8, 2011

(51) Int. Cl. A61B 5/087; A61B 5/091

(52) U.S. Cl. 600/538

(57) ABSTRACT

Systems and methods for monitoring incentive spirometry are disclosed. A patient measuring unit measures inhalation and/or exhalation flow and/or volume. The patient is instructed to follow a prescribed program of repeated inhalations and/or exhalations using the patient measuring unit. Feedback is provided to the patient to facilitate compliance with the prescribed program. All usage of the patient measuring unit is recorded and displays are provided for both the patient and an attending clinician. Prescribed performance targets are programmed, and alarms are set to indicate when prescribed targets are not met. Alarms can be set for minimum and maximum flow rates, minimum inhalation volume, minimum repeat count per set, maximum interval between sets, and maximum performance degradation compared to a recent average.

### FIELD OF THE INVENTION

One or more embodiments of the present invention relate to incentive spirometry and the prevention of pulmonary complications.

### BACKGROUND

Hospital patients and postoperative surgical patients, in particular, are at significant risk for a variety of pulmonary complications. For example, the reported incidence of postoperative pulmonary complications after upper abdominal surgery ranges from 17 to 88% [Overend 2001]. Twenty-five percent of all postoperative deaths are related directly to pulmonary complications, and in another 25% of lethal complications, there is a pulmonary component [Sabiston 2001]. On average, each postoperative infection adds $12,500 to hospital cost [Shulkin et al. 1993].

Atelectasis (partial lung collapse) is the most common pulmonary complication. Other complications include pneumonia and respiratory failure. Risk factors for postoperative pulmonary complications include previous chronic obstructive lung disease, age older than 60 years, American Society of Anesthesiology (ASA) class of II or greater, congestive heart failure, functionally dependent, prolonged surgery (more than three hours), abdominal surgery, thoracic surgery, neurosurgery, head and neck surgery, vascular surgery, aortic aneurysm repair, emergency surgery, general anesthesia, low serum albumin. The American College of Physicians recommends that all of these patients should receive some form of deep breathing exercises or incentive spirometry [Qaseem et al. 2006].

Incentive spirometry (IS) was first introduced in 1973 by Bartlett [Bartlett et al. 1973]. It has been shown to increase lung functional residual capacity (FRC). FRC decreases after surgical procedures, and this decrease is the main cause for postoperative atelectasis and pneumonia. Incentive spirometry is believed to prevent these complications by increasing the FRC.

Typical patient instructions to perform IS are to breathe in slowly (often at a target airflow rate indicated on a spirometer) until the lungs are as full as reasonable comfort allows or until a target volume indicated on the spirometer is reached. Minimum target inhaled volumes are typically set, and the spirometer indicates the actual volume achieved. While IS is now the most commonly recommended procedure to prevent post-operative respiratory complications by increasing FRC, other procedures that can also increase FRC include intermittent positive-pres sure breathing, chest physiotherapy, and deep breathing exercises. Of the various alternative therapies, IS is the least labor intensive [Qaseem et al. 2006]. Self-administration and savings in clinician labor is why IS is currently the most widely used procedure employed by hospitals to protect against atelectasis and pneumonia.

There are two different classes of incentive spirometry: volume driven and flow driven. Recently, it has been shown that volume driven devices have more favorable results [Yamaguti et al. 2010].

The typical spirometer used for IS is a disposable plastic device with a mouthpiece that can indicate both inhaled flow rate (in ml/s) and inhaled volume (in ml). The devices are purely mechanical with simple tapered tube and ball flowmeters and volume-displacement volume meters. IS devices used for the prevention of atelectasis are generally based on inhalation. Spirometers used for diagnostic pulmonary function tests, on the other hand, typically measure both inhalation and exhalation parameters. Current clinical practice for the prevention of atelectasis focuses on inhalation measurement.

In spite of its widespread use, evidence for the efficacy of IS is scant. In some studies, IS appeared to be equally effective to deep breathing, intermittent positive pressure breathing, or chest physiotherapy. In other studies, IS appeared to be only as effective as doing nothing. At present, however, IS is still widely believed to be effective, and the lack of clear supporting data for efficacy has been attributed to the absence of high-quality randomized clinical trials [Guimarãles et al. 2009].

One possible reason for the poor data supporting efficacy of IS is the lack of consistency of use of IS in hospitals. “Perhaps the principle reason that incentive spirometry is not effective is that it is not used frequently.” [Critical Care 2000] The standard IS protocol is to perform IS in sets of ten repeats every hour during waking hours. However, postoperative patients are frequently sedated and in pain. The availability of IS devices at the patient's bedside will not help the patient if they are not used as directed. As sedated and reluctant postoperative patients may not use the IS device as instructed, there is a need for someone to encourage the patient to use it as directed, and to remind the patient to continue use. Some authors even recommend that, after instructing the patient, clinicians hold patients accountable during hospital rounds for the adequacy of incentive spirometry. [Sabiston 2008, page 338] More recently, spirometers with visible markers were introduced to provide feedback to the patients, but this has not improved the outcome. As a result, even during some clinical trials, the frequency of IS was only four times daily or less, which is far less than the current clinical and manufacturer recommendations [Celli et al. 1984]. In practice, nurses on the floors are assigned to multiple patients at a time, and they are not able to monitor IS use on a regular basis. In any case, the shortcomings of current methodology are well documented [Shelledy et al. 2004].

While current clinical practice is based on manual disposable incentive spirometers, others have disclosed devices and methods to provide other kinds of patient visual feedback and encouragement such as animated figures, images which are gradually unveiled, or other patterns [Vilozni 2003], [Quinn 2009], [Boschetti-Sacco 2009]. Audio feedback has also been proposed [Means 2008], [Bryant 2010]. A non-contact IS has been described including a biofeedback system for the patient [Corn 2010], although this technique provides only a qualitative breathing waveform measurement and fails to quantify volume and flow rate, in contrast to conventional IS practice. All of these devices and methods focus on providing additional kinds of feedback to the patient and fail to provide compliance monitoring that allows clinicians to ensure that patients are following prescribed protocols and researchers to assess efficacy of those protocols.

Accordingly, there is a need in the art to encourage patients to practice IS even when they are reluctant and unsupervised. The present invention and its embodiments were conceived as a means to satisfy this need in the art, to improve patient compliance with IS protocols, and to provide a means to better evaluate the efficacy of IS in patient outcome.

### SUMMARY OF THE INVENTION

Systems and methods for monitoring incentive spirometry are disclosed. A patient measuring unit measures inhalation and/or exhalation flow and/or volume. The patient is instructed to follow a prescribed program of repeated inhalations and/or exhalations using the patient measuring unit. Feedback is provided to the patient to facilitate compliance with the prescribed program. All usage of the patient measuring unit is recorded and displays are provided for both the patient and an attending clinician. Prescribed performance targets are programmed, and alarms are set to indicate when prescribed targets are not met. Alarms can be set for minimum and maximum flow rates, minimum inhalation volume, minimum repeat count per set, maximum interval between sets, and maximum performance degradation compared to a recent average.

### BRIEF DESCRIPTION OF THE DRAWINGS

FIG. 1 shows a schematic of a monitored incentive spirometry system.

### DETAILED DESCRIPTION

### I. Introduction and Definitions

Before the present invention is described in detail, it is to be understood that unless otherwise indicated this invention is not limited to treatment of specific diseases or to specific methods of measuring airflow or volume. It is also to be understood that the terminology used herein is for the purpose of describing particular embodiments only and is not intended to limit the scope of the present invention.

It must be noted that as used herein and in the claims, the singular forms “a,” “and,” and “the” include plural referents unless the context clearly dictates otherwise. Thus, for example, reference to “a sensor” includes two or more sensors, and so forth. The invention is generally described herein with reference to exemplary embodiments where inhalation parameters are measured. While these are currently preferred embodiments for clinical practice, embodiments where exhalation parameters are measured instead or in addition are also included within the scope of the present invention. Similarly, incentive spirometry based on either volume or flow measurements or both are within the scope of the present invention even where particular embodiments are described with respect to only one of these measurements.

A “clinician” can be any person such as a nurse, physicians assistant, doctor, researcher, or other individual who is monitoring a patient's performance and compliance with a program of incentive spirometry. In a typical clinical environment, an attending physician prescribes a program of incentive spirometry, and an attending nurse programs monitoring systems and monitors patients, but other divisions of labor can be appropriate according to the specific circumstance, and these are also within the scope of the present invention. In the home use environment, the data obtained using the present systems and methods can be forwarded to the clinician reviewing and supervising the patient's performance and compliance remotely.

As used herein, the term “volume” refers to the total volume of air inhaled into (or exhaled from) the lungs in a single breath.

As used herein, the term “flow” refers to the volume rate of flow (e.g., in liters per second). Alternative terms such as “flow rate” may be used interchangeably and are intended to have the same meaning.

As used herein, the phrase “incentive spirometry” or IS refers to spirometry performed by a patient using an IS device. The IS device provides visual or auditory feedback to the patient allowing the patient to gauge his inhalation and/or exhalation volume and/or flow. IS differs from other forms of spirometry in providing data and real-time feedback regarding the patient's performance to the patient and not just to medical or laboratory personnel.

As used herein, the term “targets” refers to clinician prescribed inhalation and/or exhalation parameters for the patient to perform.

As used herein, the term “limits” refers to the extremes of the prescribed targets for inhalation and/or exhalation parameters, and can be used to trigger alarms to indicate patient noncompliance.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### II. Overview and Objectives

Embodiments of the present invention address the problems outlined in the Background section. In particular, the prior art does not adequately ascertain or measure patient compliance or performance in the use of incentive spirometry, nor does it allow one to determine if practice of IS actually prevents pulmonary complications as widely believed. Accordingly, it is one object of the invention to allow attending clinicians to monitor patient compliance and performance with respect to a prescribed IS program. It is another object of the invention to enable researchers to collect and evaluate clinical data with respect to the use of IS to determine efficacy of particular prescribed programs with respect to particular defined groups of patients.

Embodiments of the present invention allow IS use to be monitored continuously for each patient, both for frequency and adequacy. Usage is compared to pre-set minimum levels chosen by the attending physician, and if the patient does not meet those minimums, alarms are generated to alert the attending nurse. The use of alarms can help nurses and physicians to focus attention on patients who are at increased risk of developing postoperative pulmonary complications due to poor compliance with prescribed use of IS and/or inability to meet prescribed performance goals. If current belief that IS is clinically valuable is correct, this focused attention will have a significant impact on the frequency of postoperative atelectasis and pneumonia. Regardless, the continuous patient monitoring enabled by the present invention allows researchers to conduct clinical trials to evaluate the efficacy of IS protocols for various clinical indications.

### III. System Components

### A. General Characteristics

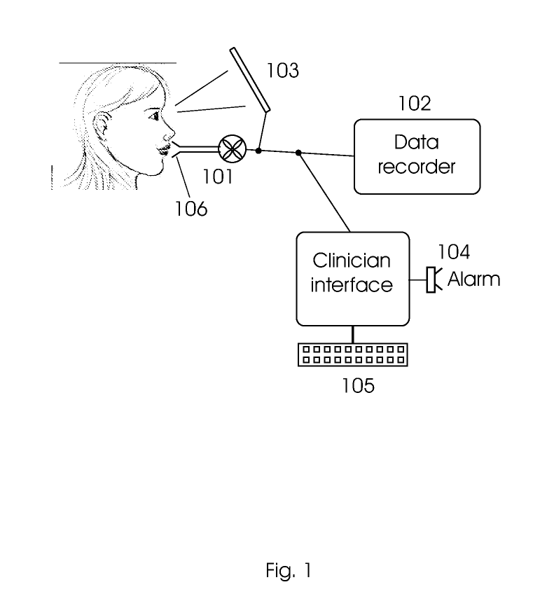

According to one or more embodiments of the present invention, a complete system, as illustrated in FIG. 1, comprises a patient measuring unit 101, wherein the measuring unit measures inhalation and/or exhalation flow rate and/or volume, a data recording device 102 capable of recording measured data from the patient measuring unit 101, a local display 103 capable of displaying the inhalation and/or exhalation flow rate and/or volume data for the patient's use, visual and/or audible alarms 104 capable of alerting a clinician when patient compliance and/or performance is outside prescribed limits; and a user interface 105 that enables a clinician to set and monitor patient usage frequency, flow and/or volume target parameters and actual performance for each use, and to set prescribed limits for the visual and/or audible alarms 104. In the clinical environment, the patient measuring unit can be a disposable unit or utilize a disposable mouthpiece 106 or disposable components, thereby minimizing microbial contamination between patients. For an outpatient environment, the patient measuring unit 101 or mouthpiece 106 may or may not be disposable, according to the desires of the patient and physician regarding cost and hygiene.

In certain embodiments, the system includes a computing machine connected to other system components. The computing machine can manage data transmission and storage, data processing, data compression, data selection, storage of demographic data and recommended clinical protocols, formatting of data for display on a user interface, a local display, and on a monitoring station, management of alarm functions, and communication with the patient and attending clinicians. Software on the computing machine can provide a count of inhalations for a set of inhalations and/or the elapsed time from the last set of measured inhalations to be shown on a local display. Similarly, the computing machine is capable of providing additional feedback to the patient corresponding to the measured data. The computing machine is capable of managing alarm functions related to the prescribed limits, for example, the prescribed minimum and maximum flow rates, minimum inhalation volume, minimum repeat count per set, or maximum interval between sets, and limits on decrease in inhalation volume relative to recent average inhalation volume.

In certain embodiments, all of the components of the system can be provided in a single device, which can be manufactured as a single unit, or manufactured as separate components and combined into a unitary device. In other embodiments, the components of the system can be provided in multiple units, in any desired combination and any number of components per unit with suitable interconnects between the units to provide data communication.

### B. Measuring Unit

The patient measuring unit can comprise any device that can detect air flow due to patient inhalation and/or exhalation and convert the measurement into an electrical signal. The unit can provide a quantitative measure of the total volume of air (e.g., in liters) inhaled into (or exhaled from) the lungs during a single inhalation cycle by the patient. The corresponding flow rate (the volume rate of flow, e.g., in liters per second) during inhalation or exhalation can also be quantitatively measured. Typically, both volume and flow are measured, although either measurement may be used alone.

There are a wide variety of instruments that are marketed as “spirometers” and used for diagnostic, educational, scientific, and medical applications to measure patient air flow on inhalation and/or exhalation. Examples can be found at any of a number of medical equipment suppliers (on-line, for example, at www.medicaldevicedepot.com/Spirometers-s/30.htm). Spirometers are basic measurement tools of any pulmonary lab, for example, where they are used to measure lung function for patients with pulmonary conditions ranging from asthma to pneumonia, emphysema, COPD, lung trauma, and so on. In the laboratory setting, spirometers with electronic readout, data recorders, and computer interfaces are common to support the needs of a variety of clinical and research uses. Spirometers can measure and record during both inhalation and exhalation, and can present the resulting data in a variety of useful ways to characterize pulmonary function. Any of a variety of well-known airflow measurement techniques can be used with varying tradeoffs in cost and accuracy. While diagnostic spirometers could be used for IS, they are designed and intended for infrequent single-measurement applications, and are generally too expensive and too few in number in hospital settings to allow them to be made available to patients for hourly use. Further, diagnostic spirometry has typically focused on exhalation measurements.

The patient measuring unit can be disposable, or can comprise both disposable elements and non-disposable elements. IS is typically practiced using a disposable and entirely mechanical device with visual indicators to allow the patient to see measured flow and/or volume for the current breath. Common volume-focused devices indicate inhaled volume (in ml; range 0 to 2500, 4000, or 5000 ml) by piston displacement. Flow rate is also indicated, either using word descriptors (“high, medium, low”) or quantitatively (in ml/s), typically using a tapered tube and ball flowmeter. Examples include the VOLDYNE® volumetric exercisers made by Hudson RCI, incentive spirometers made by Medline Industries, Inc., and AIRLIFE® incentive spirometers made by Allegiance.

According to one or more embodiments of the present invention, the patient measuring unit can comprise any air flow or volume measuring device. In certain embodiments, the patient measuring unit comprises a mechanical incentive spirometry device such as the incentive spirometers described above. Additional sensors are added to transduce the flow and/or volume measured into electrical signals (e.g., by measuring the position of the flow and/or volume indicators). These additional sensors can be incorporated directly into the disposable unit or can be non-disposable but detachable from the disposable unit. Any suitable sensor technology can be used including optical sensors, linear transducers such as variable resistors, mechanical switches, etc.

In other embodiments of the present invention, the patient measuring unit can comprise a mouthpiece, tube, and a flowmeter capable of generating an electrical signal. Any suitable flowmeter technology can be used. Examples include rotating cups, rotating vanes, hot-wire anemometers, laser doppler, ultrasonic, deflecting ball, pressure plate, and pitot tube flowmeters. A rotating cup or vane flowmeter can include, for example, a simple optical interrupt sensor to count rotation speed to generate an electrical signal (pulse train) whose frequency is related to flow rate, or alternatively any sensor means capable of measuring the angular speed of the cup or vane. Optionally, the rotational speed sensor can be either part of, or separate from, the patient measuring unit. Similarly, using other flowmeter technologies, selected portions of the flow sensor can be included in the patient measuring unit, or the entire sensor can be included. Volume measurements can be obtained by integrating the flow data over time, or measured separately by any suitable volume measurement technology. Hybrid systems incorporating both conventional mechanical spirometers with mechanical visual indicators and separate sensors capable of generating an electrical signal can also be used.

Commercially available laboratory spirometers can also be used in embodiments of the present invention by interfacing and transmitting available digital or analog representations of spirometry data to a system for monitoring and recording patient data.

### C. Data Recording

According to one or more embodiments of the present invention, the electrical signal from the patient measuring unit is connected to a data recording device such as a chart recorder, digital data-logger, or general-purpose computing machine including both local machines and remote servers. The general-purpose computing machine can comprise any type of computer such as but not limited to desktop computers, laptop computers, tablet computers, and handheld devices including cellular telephone devices. The computing machine can be combined with the system at manufacture, or it can be provided separately. If provided separately, software to enable operation of the system would be provided and installed separately by a user. In certain embodiments data can be stored directly into an electronic patient chart or record. Any available storage medium can be used including semiconductor memory, magnetic or optical disk drives, tape drives, etc. The computer can be dedicated to the application or can be any available computing device that can be readily connected to the patient measuring unit. Data can be preprocessed to reduce overall data capacity requirements. For example, for each inhalation, the start time, duration, average flow rate, and total inspired volume can be recorded.

### D. Local Display

According to one or more embodiments of the present invention, the patient measuring unit does not include a patient display of any sort but only provides an electrical signal. The local display can be integral to the patient measuring unit, or can be separate. Local display of measured data can be provided such that the data is available to the patient, or can be provided such that the data is only available to the clinician. A separate patient display can be provided to indicate flow and/or volume for the current inhalation by processing and displaying data from the electrical signal in numerical and/or graphical form. Target values can also be displayed to help the patient visualize current performance vs. goal performance. The patient display can also provide a count of inhalations for the current set of inhalations as well as the elapsed time from the last set of measured inhalations.

In certain embodiments, the system can include additional patient feedback means, for example, visual and/or audio feedback such as those described by [Vilozni 2003], [Quinn 2009], [Boschetti-Sacco 2009], [Means 2008], [Bryant 2010], and [Corn 2010] in addition to the local display of performance in numerical or graphical form. These feedback means can be especially useful for patients with disabilities or conditions that limit their ability to see and understand numerical or graphical presentations of performance, for example to provide audio assistance to patients with sight disabilities. Other patients that can benefit from auxiliary feedback means include, for example, young children and patients with reduced mental capacity.

According to one or more embodiments of the present invention, the local display of measured data can be provided on the standard bedside patient monitor used to display routine patient data (such as heart waveforms, pulse rate, breathing rate, O_{2 sat}, and/or blood pressure).

### E. Monitoring Station

Alternatively, or in addition, the measured data can also be transmitted to a monitoring station such as the nurses' station for a hospital ward or workstation for an attending physician. For example, a monitoring station located at a nurses' station can comprise the clinician interface 105, data recorder 102, and alarm 104. Data can be transmitted via any standard communications channels such as wired or wireless networks (e.g., ethernet) or a serial interface or custom wireless link. Optionally, the bedside patient monitor display can also be used to implement the separate patient display described immediately above.

For home use, data can be transmitted over a suitable available communications channel such as the internet or a land-line, wireless, or satellite telephone to a monitoring station. An example of a monitoring station for use with home-based incentive spirometry is an independent monitoring station provided by an equipment manufacturer or service company similar to the monitoring stations that are provided for medical alert devices, cardiac monitors, and the like. Measured data can be forwarded periodically to the office of the patient's physician, and more immediate response can be provided for any alarm conditions. Such a response can include both telephone consultation with the patient or text messaging to the patient (for example, to remind the patient to use the incentive spirometer as instructed), and notification of the attending physician (for example, to provide notification of declining patient performance).

### F. User Interface

The system for monitoring incentive spirometry also comprises an electronic user interface, such as the clinician interface 105 in FIG. 1, that enables a clinician to monitor the patient's practice of IS. The user interface allows the clinician to prescribe targets for patient usage frequency, flow and/or volume parameters for each use, and to set alarm limits for alarms capable of alerting a clinician when patient compliance and/or performance does not meet prescribed minimum standards. Certain defaults can be preset and suggested based on standard clinical practice, patient demographics, patient status with respect to specific recent surgical history or other disease condition. The clinician can override any specific presets according to the needs of the particular patient. Adjustable settings can be presented to the clinician in tabular (numerical) form and/or via suitable graphic equivalents such as slide bars or scroll bars. Performance data can similarly be presented and summarized in various numerical and graphical formats. Alarm states and conditions can similarly be represented using any suitable combination of numerical, graphical, and audible formats. For example, any condition that generates an alarm can result in both a visual icon and audible alarm together with a numerical readout that persists on the user interface until explicitly reset by an attending clinician. Computational support for data processing and user interface management can be provided by the same or a different computing machine from that which manages the data recording function.

### IV. Methods

According to one or more embodiments of the present invention, a patient is instructed to follow a prescribed program of repeated inhalations and/or exhalations such as that given in Example 1 using a patient measuring unit as described above. The predicted inspiratory capacity of a healthy individual is based on sex, age and height and is available for different ethnic groups [Polgar and Weng 1979]. Current clinical practice is generally in the range of 1500-2500 ml; older literature such as [Peruzzi and Smith 1995] recommends 14 ml/kg or 1120 ml for an 80 kg adult patient. Measured data is displayed locally together with additional incentive patient feedback as desired. Measured data is also recorded and displayed to an attending clinician who can set prescribed targets for patient performance such as those given in Example 1.

According to one or more embodiments of the present invention, a method is provided for monitoring incentive spirometry comprising providing a patient measuring unit, wherein the patient measuring unit measures inhalation and/or exhalation flow and/or volume; providing a local display capable of displaying inhalation and/or exhalation flow rate and/or volume; instructing a patient to follow a prescribed program of repeated inhalations and/or exhalations using the patient measuring unit; setting prescribed targets for patient performance; setting alarms to indicate when prescribed targets are not met; recording measured data; and displaying measured data to an attending clinician.

In certain embodiments, the local display is integral to the patient measuring unit, while in other embodiments, the local display is separate from the patient measuring unit. The method can comprise providing additional feedback to the patient corresponding to the measured data. The method can further comprise transmitting measured data to a monitoring station.

In particular embodiments, the prescribed targets for patient performance comprise minimum and maximum flow rates, minimum inhalation volume, minimum repeat count per set, or maximum interval between sets. The prescribed targets for patient performance can further comprise limits on decrease in inhalation volume relative to recent average inhalation volume.

The patient measuring unit can be disposable or can comprise both disposable elements and non-disposable elements.

The attending clinician can also set and monitor alarms which indicate when prescribed targets are not met. Examples of alarms that can be set and monitored are also given in Example 1. The clinician can be prompted to attend to the patient when alarms are triggered, by reminding the patient to continue to practice the spirometry, or by determining if the patient is in need of further medical attention.

In summary, the inventor has identified the problem of under-usage of incentive spirometry in hospitals and addresses it in a novel way by providing an incentive spirometry system with electronic monitoring, data processing, and alarms to achieve complete patient supervision while guiding the limited nursing resources to the patients that need that help the most.

While embodiments of the present invention can be used in a wide variety of clinical and research applications wherein different targets and alarm levels and responses may be appropriate, examples will now be described for specific applications.

### Example 1

### Incentive Spirometry for Surgical Patients

A typical scenario is for a patient in a hospital ward recovering from major surgery of the chest or abdomen. As previously discussed, such patients are frequently at significant risk of pulmonary complications during their postoperative recovery. The following program is prescribed by the patient's physician:

The measurement unit should have a typical measurement range of about 0-4000 ml. The patient is instructed to perform sets of ten deep inhalations repeated at hourly intervals during waking hours where each inhalation should take 4-12 seconds (alternatively definable as a flow rate of, e.g., 200-600 ml/sec) and achieve a target inhalation volume of at least 1500-2500 ml. The patient is instructed to keep the lungs full at maximum capacity for 1-2 seconds. Performance expectation may also be downgraded depending on the clinical status of the patient. Patient performance can be displayed on a local display to assist the patient in understanding and achieving target performance.

In this example, the waking hours (e.g., 7 am to 9 pm) would be set, together with the number of repeats per set (10), the target time interval between sets (1 hr), target inhalation rate (in either seconds per inhalation or ml/sec), and target inhalation volume. In addition to monitoring, displaying, and recording actual patient use of the IS device, alarms are generated for parameters outside prescribed limits. Some non-limiting examples of alarms that can be generated include the following: inhalation rate too fast or too slow, inhalation less than 75% of target (e.g., less than 1875 ml if the target is set at 2500 ml), fewer than target repeat count (e.g., fewer than 10 inhalations recorded over a three minute interval after a set begins), more than 1.5 times the target time interval between sets (e.g., more than 1.5 hours since last set), decline of more than 20% in inhalation volume (optionally averaged over a group such as one set) compared to average for previous day, and so forth. Each of these alarms can be adjusted to suit clinical needs and can be disabled if not desired. The attending nurse responds as appropriate, for example, by simply resetting the alarm if the patient is away for a procedure or asleep, reminding the patient that it is time for another set, repeating the instructions on how to perform a set, asking the patient if there is a problem (e.g., excessive pain), or alerting the attending physician of a decline in patient condition that requires action. Instructions for nurses can also be included at the physician's direction. Certain conditions can also be set to generate automatic paging of an attending clinician if desired. All measured data and alarms can be displayed on a monitor at the clinician interface and optional monitoring station, and recorded in an electronic patient chart (if used).

### Example 2

### Monitoring Incentive Spirometry in Patients with Chronic Lung Disease

As with postsurgical patients, there is a need for monitoring compliance and measuring efficacy of IS for patients with chronic lung disease who did not necessarily have surgery. In this group of patients, IS is believed to improve arterial blood gases and health-related quality of life in patients with Chronic Obstructive Pulmonary Disease (COPD), even though it may not be observe to alter pulmonary function parameters. [Basoglu et al. 2005] A program of IS can be prescribed for either an inpatient or outpatient setting as appropriate. Target performance and alarm setting can be similar to those for the postsurgical patients of Example 1, although there may be more need for individual adjustment according to the residual lung function and therapeutic needs of these patients.

In addition, any degradation in performance can be noted and flagged early in order to provide further treatment before the patient's condition deteriorates further. For outpatient use, the system embodied in a patient unit can be useful for monitoring in home IS, and can report performance frequency and pulmonary function to the monitoring clinician from the remote location, thereby alerting clinicians to the patient's need for assistance.

### Example 3

### Clinical Trial Utilizing Monitored Incentive Spirometry

Embodiments of the present invention can also be used to enable clinical trials to be conducted for determination of efficacy of IS programs to prevent pulmonary complications. A cohort of patients undergoing thoracic or abdominal surgery is recruited. The cohort is divided into two arms matched for surgery type, gender and age: one arm (control) receives standard hospital post-operative care (excluding explicit respiratory therapy); the second group (IS) receives IS with monitoring both during hospitalization and continuing until three weeks after surgery as described in Example 1.

Primary outcome measures include reductions in episodes of community acquired pneumonia (CAP) and related severe respiratory complications (atelectasis) during the surgical recovery period (three weeks after surgery).

Secondary outcome measures include reductions in hospitalizations due to a primary diagnosis of respiratory complications during the surgical recovery period (three weeks), duration of respiratory illness and hospitalization during the surgical recovery period (three weeks), impact of respiratory complications on quality of life during the surgical recovery period (three weeks).

Inclusion criteria include an impaired ability to cough (cough peak flow less than 300 L/min), oxygen saturation greater than or equal to 95% when awake and not receiving supplemental oxygen, end-tidal carbon dioxide level less than 43 mm Hg.

Exclusion criteria include under 18 years of age, currently have a tracheotomy tube, lung disease as seen on chest x-ray that results in a baseline oxygen saturation decreasing below 95% during daytime hours and cannot be normalized by usual way of coughing, have a significant medical complication and psychiatric condition that would interfere with the conduct of the study or interpretation of the study results.

Patient recovery and post-surgical complications such as infection, pleural effusion, and atelectasis in the two groups are noted and recorded. The efficacy of IS in preventing respiratory complications can be determined by statistical analysis of patient outcome in the control vs. intervention arms.

It will be understood that the descriptions of one or more embodiments of the present invention do not limit the various alternative, modified and equivalent embodiments which may be included within the spirit and scope of the present invention as defined by the appended claims. Furthermore, in the detailed description above, numerous specific details are set forth to provide an understanding of various embodiments of the present invention. However, one or more embodiments of the present invention may be practiced without these specific details. In other instances, well known methods, procedures, and components have not been described in detail so as not to unnecessarily obscure aspects of the present embodiments.

### VI. References

### All of the Following References are Incorporated Herein by Reference in their Entireties

[Bartlett et al. 1973]: Bartlett RH, et al., “Respiratory maneuvers to prevent postoperative pulmonary complications, a critical review,” JAMA 224(7), 1017-21, 1973.
[Basoglu et al. 2005]: Basoglu O K et al., “The efficacy of incentive spirometry in patients with COPD,” Respirology 10(3), 349-53, 2005.
[Boschetti-Sacco 2009]: Boschetti-Sacco, P; U.S. Patent Application Publication No. 2009/0184968.
[Bryant 2010]: Bryant, TK; U.S. Patent Application Publication No. 2010/0121211.
[Celli et al. 1984]: Celli BR, et al., “A controlled trial of intermittent positive pressure breathing incentive spirometry and deep breathing exercises in preventing pulmonary complications after abdominal surgery,” Am Rev Respir Dis; 130(1): 12-15, 1984.
[Corn 2010]: Corn, SB; U.S. Patent Application Publication No. 2010/0305466.
[Critical Care 2000]: Textbook of Critical Care, 4^{th} Edition, ed. by Vincent J-L et al., Elsevier Saunders, 2000, p. 1324.
[Guimarãles et al. 2009]: Guimarãles MMF et al., “Incentive spirometry for prevention of postoperative pulmonary complications in upper abdominal surgery,” Cochrane Database Syst Rev. July 8; (3), CD006058, 2009.
[Means 2008]: Means, MJ; U.S. Patent Application Publication No. 2008/0082139.
[Overend et al. 2001]: Overend TJ, et al., “The effect of incentive spirometry on postoperative complications: a systematic review,” Chest 120(3), 971-8, 2001.
[Peruzzi and Smith 1995]: Peruzzi WT and Smith B, “Bronchial hygiene therapy,” Crit. Care Clin. 1995: 11(1), 79-96, 1995.
[Polgar and Weng 1979]: Polgar G and Weng TR, “The functional development of the respiratory system from the period of gestation to adulthood,” Am Rev Respir Dis. 120(3), 625-95, 1979.
[Qaseem et al. 2006]: Qaseem A et al., “Risk assessment for and strategies to reduce perioperative pulmonary complications,” Ann Intern Medi, 144, 575-80. 2006.
[Quinn 2009]: Quinn, D E; U.S. Pat. No. 7,625,345.
[Sabiston 2001]: Sabiston Textbook of Surgery, 16th Edition, Townsend CM Jr, et al., p. 205, Saunders, 2001.
[Shulkin et al. 1993]: Shulkin DJ et al., “The economic impact of infections. An analysis of hospital costs and charges in surgical patients with cancer,” Arch Surg 128(4), 449-52, 1993.
[Shelledy et al. 2004]: Shelledy D C et al., “An assessment of appropriateness of respiratory care delivered in a 450-bed acute care Veterans affairs hospital,” Respir Care, 49(8):907-16, 2004.
[Vilozny 2003]: Vilozni, D; U.S. Pat. No. 6,508,772.
[Yamaguti et al. 2010]: Yamaguti WPS et al., “Diaphragmatic mobility in healthy subjects during incentive spirometry with a flow-oriented device and with a volume-oriented device,” J Bras Pneumol 36(6), 738-45, 2010.

What is claimed is:

1. A system for monitoring incentive spirometry comprising:
a patient measuring unit, wherein said patient measuring unit measures inhalation and/or exhalation flow rate and/or volume;
a data recording device capable of recording measured data from said patient measuring unit;
a local display capable of displaying said inhalation and/or exhalation flow rate and/or volume;
visual and/or audible alarms capable of alerting a clinician when patient compliance and/or performance is outside prescribed limits; and
a user interface that enables a clinician to set and monitor patient usage frequency, flow and/or volume parameters for each use, and to set said prescribed limits for said visual and/or audible alarms.

2. The system of claim 1, wherein said local display is integral to said patient measuring unit.

3. The system of claim 1, wherein said local display is separate from said patient measuring unit.

4. The system of claim 1, wherein said local display provides a count of inhalations for the current set of inhalations and/or the elapsed time from the last set of measured inhalations.

5. The system of claim 1, further comprising means for providing additional feedback to said patient corresponding to said measured data.

6. The system of claim 1, further comprising means for transmitting measured data to a monitoring station.

7. The system of claim 1, wherein said prescribed limits comprise minimum and maximum flow rates, minimum inhalation volume, minimum repeat count per set, or maximum interval between sets.

8. The system of claim 7, wherein said prescribed limits further comprise limits on decrease in inhalation volume relative to recent average inhalation volume.

9. The system of claim 1, wherein said patient measuring unit is disposable.

10. The system of claim 1, wherein said patient measuring unit comprises both disposable elements and non-disposable elements.

11. A method for monitoring incentive spirometry comprising:
providing a patient measuring unit, wherein said patient measuring unit measures inhalation and/or exhalation flow and/or volume;
providing a local display capable of displaying said inhalation and/or exhalation flow rate and/or volume;
instructing a patient to follow a prescribed program of repeated inhalations and/or exhalations using said patient measuring unit;
setting prescribed targets for patient performance;
setting alarms to indicate when prescribed targets are not met;
recording measured data; and
displaying measured data to an attending clinician.

12. The method of claim 11, wherein said local display is integral to said patient measuring unit.

13. The method of claim 11, wherein said local display is separate from said patient measuring unit.

14. The method of claim 11, further comprising providing additional feedback to said patient corresponding to said measured data.

15. The method of claim 11, further comprising transmitting measured data to a monitoring station.

16. The method of claim 11, wherein said prescribed targets for patient performance comprise minimum and maximum flow rates, minimum inhalation volume, minimum repeat count per set, or maximum interval between sets.

17. The method of claim 16, wherein said prescribed targets for patient performance further comprise limits on decrease in inhalation volume relative to recent average inhalation volume.

18. The method of claim 11, wherein said patient measuring unit is disposable.

19. The method of claim 11, wherein said patient measuring unit comprises both disposable elements and non-disposable elements.

20. The method of claim 11, wherein a clinician is prompted to attend to the patient when said alarms are triggered.
